(19) United States
(12) Patent Application Publication
(10) Pub. No.: US 2007/0292959 A1
(43) Pub. Date: Dec. 20, 2007

(54) Sample Age Monitoring Devices And Methods

(76) Inventors: Paul Laurence Cervi, Essex (GB); Anthony Sidney Everitt, Essex (GB)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/578,680

(22) PCT Filed: Nov. 18, 2004
(86) PCT No.: PCT/GB04/50028

§ 371 Date: Apr. 30, 2007

Related U.S. Application Data
(60) Provisional application No. 60/530,894, filed on Dec. 22, 2003.

(30) Foreign Application Priority Data
Dec. 8, 2003 (GB) 0328391.8

(51) Int. Cl. G01N 33/48; B01L 9/00; G01N 21/00; G01N 21/01

(52) U.S. Cl. 436/63; 422/104; 422/55; 436/164

(57) ABSTRACT

A sample age monitor for monitoring the age of a biological sample, said sample age monitor comprising a release device to provide an age indicator into a medium preferably comprising the sample substantially predictably such that an age estimate of the sample is determinable by measuring a level of the age indicator. A biological sample holder including the sample age monitor is also described. The device may be employed to determine the age of a blood sample, and hence whether the results of lab analysis of the sample are reliable. In one embodiment the release device comprises a container holding a storage medium storing the age indicator. The age indicator may comprise a metal, in particular lithium, ion.

This invention is generally concerned with apparatus and methods for monitoring and/or estimating the age of biological samples, in particular blood samples.

When taking a blood sample from a human or animal body for laboratory assay it is important to ensure that the sample has not aged beyond the point in time where the assay has been validated as reliable or accurate. In many situations the duration of storage and transport of samples can affect the results obtained from an analysis. The following examples illustrate some of the problems which can arise.

Measurement of blood potassium is one of the commonest blood tests performed. Only a small proportion of the total body potassium is contained in the blood plasma, 98% being located within the cells of the body including the red blood cells. Within the body there is a constant tendency for potassium to diffuse into the extra cellular fluid and therefore, in the living body, a mechanism exists to maintain this high concentration of potassium in body cells. Blood specimens taken from patients within a hospital generally reach the laboratory within one hour but specimens from general practice or peripheral hospitals may not reach the laboratory until the following day. Once a blood sample has been taken, potassium begins to leak out of the blood cells and this means that by the time the blood sample is tested, the amount of potassium which was originally in the plasma may have changed significantly. Results of such blood tests are therefore no longer clinically meaningful and consequently it is recommended in general that no more than three hours elapse before samples are centrifuged, (which is the initial process whereby the samples are stabilised before analysis). It would therefore be advantageous to a laboratory to have an independent means of ensuring that samples have not aged beyond, say, three hours before centrifugation—that is, not simply relying upon records attached to the sample, which may not be accurate.

Measurement of the coagulation status of blood is another common laboratory investigation. The assay is generally sensitive to the storage condition of the blood sample before centrifugation as the coagulation cascade may be activated by exposure to the cold (so called “cold activation”) or prolonged delay before assay. It is therefore recommended that no more than three hours elapse between the time of talking of the sample and centrifugation—and it would therefore be advantageous to have some way of independently validating the age of a sample before analysis.

Routine counting of blood cells (red cells, white cells and platelets) when using current preservatives and counting methods is not as susceptible to pre-analytical factors as the chemical analyses described above but occasionally samples are delayed before arriving in the laboratory, and samples older than 24-48 hrs should be rejected or qualified as being unreliable.

Many specialist assays of hormonal metabolites or short lived intermediary compounds, such as in immunoassays and cytokine assays, are highly dependent on pre-analytical storage conditions—and once again it is desirable that independent means exist to confirm that the assay is valid,

In an attempt to overcome these problems, various approaches have been used:

Accurate and correct documentation of sampling and handling procedures:

However such documentation is often incomplete or inaccurate due to the high number of samples routinely undertaken.

Providing sites where blood is taken with a centrifuge so that they can separate the serum soon after the blood is taken. However suitable safe centrifuges are very costly and this approach requires non-laboratory staff to be trained in tile technique, including safety and quality control aspects.

Equipping sites with analytical equipment and training staff to perform the assay themselves. However this is even more costly in terms of providing specialist equipment, and its proper maintenance and operation by skilled staff. This method is not suitable for all the analyses which may be required.

Opening central laboratories for the handling of specimens during the evening. Again, however this is an expensive solution requiring extra transport from all peripheral sites to the central laboratory and incurs increased staffing costs.

There therefore exists a need for a simple, reliable and inexpensive way of determining the age of a blood or other biological sample. One method for determining the useable life or age of biological fluids by adding a known amount of radioactive isotope to a predetermined quantity of such a fluid is described in GB1,001,875 but there are safety concerns with such a technique. A method of determining age of blood traces by spectrometric analysis of reflected artificial light is described in DE19811142. A timer for an automatic parking coupon comprising a porous wick which soaks up liquid from a reservoir to create a visible trace is described in WO 91/04520. The citations against WO 91/04520 describe other wick-based timing devices. Examples of background prior art relating to control release drug delivery devices are described in EP0 621 032A, WO02/07619, and WO01/32149, Further background prior art call be found in EP0 784 201 and in U.S. Pat. No. 5,679,577.

According to a first aspect of the present invention there is provided a sample age monitor for monitoring the age of a biological sample, said sample age monitor comprising a release device to provide an age indicator into a medium substantially predictably such that an age estimate of said sample is determinable by measuring a level of said age indicator.

The biological sample preferably comprises a liquid, in particular an aqueous liquid such as blood into which the age indicator is released. The release device preferably comprises a container holding a storage or carrier medium or matrix for providing said age indicator The age indicator may be stored in said carrier medium or the carrier medium may hold a substance which, in conjunction with said biological sample, provides said age indicator. Thus the age indicator may comprise a pre-cursor to the age indicator, or an enzyme or catalyst, or some other substance which is converted into or releases the age indicator on contact with said biological sample. In a preferred embodiment the age indicator comprises a metal ion, in particular a lithium ion and in this case the storage medium may store a lithium salt or complex, in other embodiments a dye may be used as an indicator.

The container of the release device is preferably formed from a substantially inert material such as glass or silica. Where the sample comprises a blood sample the release device as a whole is preferably of a greater density than the blood plasma, more preferably of a greater density than cells, especially red blood cells, of the sample. In this way when the blood sample is centrifuged prior to analysis the release device may be substantially separated from the blood plasma so that release of the age indicator into the plasma is diminished, and preferably substantially halted, providing a well-defined end point to the age estimate.

The container preferably includes an indicator release portion which may comprise a porous or semi-porous plug or region of the container, and/or a semi-permeable membrane, and/or one of more capillaries, and/or an opening to allow the age indicator (for a pre-cursor or catalyst for other generator therefor) into the sample by diffusion or osmosis. Thus the container may comprise a small glass tube open at one end, preferably of a volume less than 100 μl, more preferably less than 50 microliters or approximately 20 microliters in capacity. In embodiments, however, the container may be omitted, for example where the storage medium comprises a biodegradable material such as a biodegradable polymer. Preferably however, the release is predictable to such an extent that an age estimate accurate to better than 30 minutes, more preferably better than 10 or 15 minutes, over a six hour period is obtainable. It will be recognized, however, that depending on the application a precise estimate of age may not be necessary—it may suffice, for example, simply to determine whether a sample is above or below a permitted threshold age, say for analysis,

In another aspect the invention provides a controlled release device for determining the age of a blood sample, the device being configured to release an age indicator into the blood sample on contact with the sample, and to substantially cease release of said age indicator into plasma of said blood sample after centrifugation of said sample.

It will be recognized that the release of the age indicator need not necessarily be a steady or controllable release. For example, a calibration curve may be employed in the case of a non-linear release rate over time to relate a particular level of age indicator to a particular sample age (optionally for a particular sample type).

The invention also provides a biological sample holder including sample age monitor or controlled release device as described above. The biological sample holder may comprise, for example, a blood sampling tube and the sample age monitor/controlled release device may then determine duration of time between addition of a liquid sample to the tube and centrifugation of the sample or the performance of a laboratory test analysis.

Thus in a further aspect the invention provides a biological sample holder including a timer mechanism to determine a duration of time between addition of a sample to said holder and centrifugation or laboratory analysis of the sample.

The invention also provides a biological sample holder including a chemical timer mechanism.

The invention also provides a method of determining an age for a biological sample, the method comprising: containing the sample after collection together with an age indicator release device; and determining a level of said age indicator to determine said age.

The age indicator release device may be added to the sample after collection or the sample may be collected into a container in which the release device is already present. Preferably the device comprises a container or enclosure configured to define an opening or release portion for releasing the age indicator.

As previously mentioned the age need not be an estimate of a particular sample age but may simply a determination of whether the sample age is above or below a threshold age for analysis of the sample.

These and other aspects of the present invention will now be further described, by way of example only with reference to the accompanying figures in which:

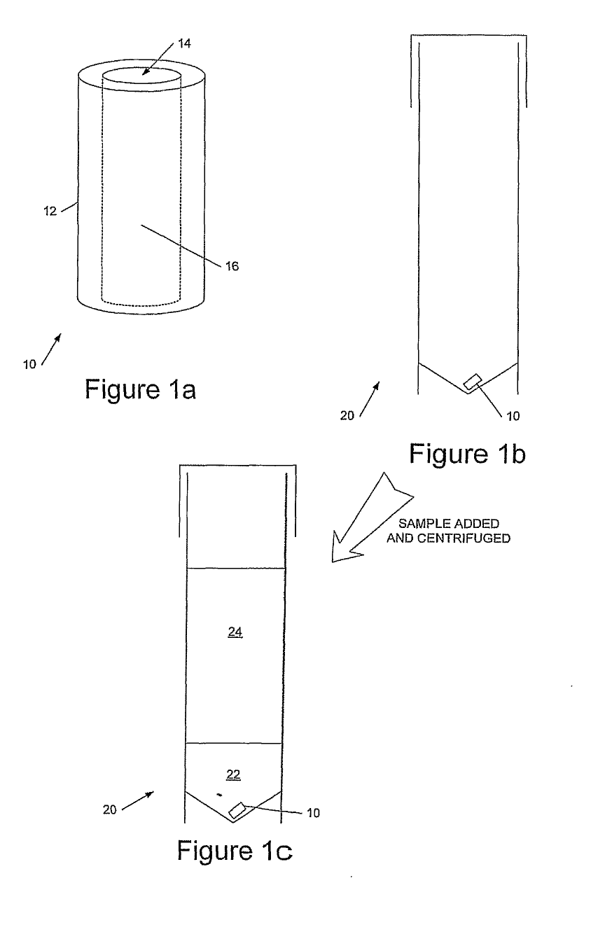

FIGS. 1a to 1c show, respectively, a blood sample age monitoring device, a vertical cross-section through a blood sampling tube including the sample age monitoring device of FIG. 1a, and the sample tube of FIG. 1b including a sample after centrifugation; and

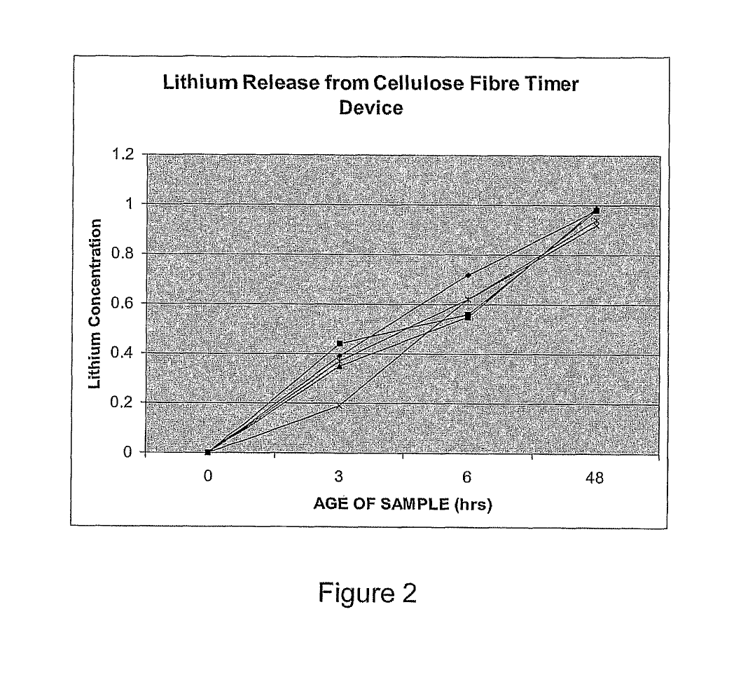

FIG. 2 shows a graph of lithium concentration against time for an embodiment of the invention.

In those assays performed in serum or plasma, the blood sample is centrifuged on arrival in the laboratory. The process of centrifugation separates the cellular components (which sediment at the bottom of the sampling tube) from the solution (or supernatant), and it is at this point in time that the most analytes in the supernatant become stable. The age of the blood sample is therefore best defined as the time between the taking of the blood sample and the time of centrifugation. (hi case of analyses performed on whole blood (for instance full blood counts (FBC), the age of the sample may be defined as the time between obtaining the sample and the time of the analysis). Embodiments of the present invention add a timing device to the blood sampling tube, configured such that upon the addition of the liquid sample, a time dependent reaction is initiated, which is terminated on either centrifugation of the sample or performance of the analysis, whichever occurs sooner. Preferred embodiments should not interfere with the intended analysis to be performed.

Thus broadly speaking one aspect of the invention may be conveniently defined as a method of adding a timing device to a blood sampling tube, which is activated by exposure to liquid and which is terminated by centrifugation or performance of the analysis.

In one preferred embodiment, the timing device comprises a small container or tube which is closed at one end, for example a miniature test tube (MTT), The MTT contains a precise amount of indicating substance (IS) which is released from the MTT in a rate dependent manner on contact with a liquid phase.

Referring now to FIG. 1a, this shows a blood sample age monitoring device 10 comprising a miniature glass tube 12, sealed at one end and having an opening 14 at the other end. Inside the tube is a medium 16 in which an indicating substance is incorporated. When the monitoring device is exposed to blood the indicating substance diffuses out over time through opening 14. It is therefore preferable that opening 14 is of substantially uniform size from device to device so that a time measurement based upon the level of indicating substance in the blood sample can be accurately calibrated.

Conveniently a sample age monitoring device 10 may be included in a biological sample holder such as blood sampling tube 20 of FIG. 1b, provided that the sample age monitor is stable when stored in a dry condition (which is true of embodiments described below). In this way medical personnel need not add an age monitor to a sample, thus simplifying procedures.

FIG. 1c shows the result when a blood sample is added to the sampling tube 20 of FIG. 1b and centrifuged. As can be seen the blood sample separates into two main fractions, a portion 22 comprising mainly red blood cells and a portion 24 comprising mainly plasma. Since the red blood cells have a greater density than the plasma these lie at the bottom of the tube, and since in preferred embodiments the age monitor 10 has a greater density still, this lies at the very base of the tube, under cells 22. In this way it is substantially separated from plasma 24, thus reducing and effectively ceasing egress of the indicating substance to the blood plasma 24. -II this way centrifugation effectively halts the timing mechanism of the sample age monitor 10.

Some aspects of preferred embodiments of the sample age monitor will now be further described, in further detail.

The miniature test tube can be manufactured from an inert substance such as glass or metal. The material is preferably chosen to be of relatively high density (greater than cells of the blood sample), such that on centrifugation, it will be deposited with its contents at the bottom of the sample container. The miniature test tube dimensions are preferably very precise and uniform—such that there is minimal variation in size from one miniature test tube to the next. The size and shape of the miniature test tube open end is an important dimension and should be kept uniform, for example to better than 1%, from one miniature test tube to the next, as this regulates the rate at which the age indicator is released from the miniature test tube. In other embodiments the container may be omitted and the age indicating substance (described below) may be incorporated into a slow-release tablet.

The indicating substance in one preferred embodiment is the lithium metal ion. This is because many chemistry analysers are already capable of measuring this compound with little additional effort. In addition, it is a small molecule, and will readily diffuse from the miniature test tube in a predictable maimer. It does not interfere with the measurement of other commonly analysed substances and it is rarely requested routinely (except for cases of patients talking Lithium treatment, where drug levels are being checked, or in cases of suspected toxicity).

Alternative indicating substances may be chosen, for example a dye of known spectral absorption. Once again this will be released in a predictable manner from the miniature test tube. The analyzing instrument can be configured to automatically read the absorption at a specified dye absorption wavelength, and this will vary with and preferably be proportional to the age of the sample. A wide range of dyes are potentially suitable and, for example, methylenie blue has given good results, A dye is useful where a sample is to be analysed by an instrument without the ability to measure metal ions, for example coagulation analysers and FBC (full blood count) analysers, but which can be configured for calorimeter analysis. In such circumstances the dye should be chosen such that its absorption does not significantly interfere with other analysis of the sample.

Another alternative is to choose an indicating substance which makes use of a substrate to product transition with or without an enzyme/catalyst dependence. For example a timer may comprise a substrate (a material or substance on which an enzyme or catalyst acts), of which there is a large supply, and a catalyst/enzyme, which is rate limiting. On exposure to liquid, the timer reaction is initiated, for example because the two constituents are contained in separate dissolving tablets on the addition of liquid, and the product is delivered into the blood sample in a predictable manner. Preferably the concentration of the product is proportional to the time of exposure to the liquid. One example is the peroxidase reaction (which the skilled person will know), where a specific substrate (say Di-Amino-Benzedrine DAB) is converted to a brown product by the action of the peroxidase. This product can be measured spectrophotometrically by an analysers. There are innumerable enzymes/substrate options but one drawback with enzyme reactions is that they tend to be very temperature dependent and blood samples in transit (depending on the season etc) can be exposed to a large range of temperatures (2° C. to 37° C.).

Other indicating substances, such as molecules not normally measured in routine analysis, may also be chosen as indicating substances.

The medium in which the indicating substance is incorporated is important in that it should preferably have the following properties: The medium should be stable when stored for prolonged periods of time (say several months), either at room temperature, or refrigerated (4-8° C.); it should allow diffusion of the indicating substance into the sample on contact with an aqueous sample; it should be semi-permeable to the indicating substance molecules; the rate of diffusion of the indicating substance from the medium should be relatively constant and relatively temperature independent in the range 2 to 35° C.; and it should not degrade if it becomes dehydrated. In a preferred embodiment, the medium is composed of cellulose fiber, or complex carbohydrate (starch) which is highly absorbent to an aqueous medium, and which is stable when allowed to dry. Alternative media which may be employed include other hydrocolloid/aqueous mixtures, examples of which include naturally occurring compounds such as agar or gelatine gels, gum arabic gels, and synthetic gels such as poly vinyl alcohol. Other carrier media which may potentially be employed are described in EP 1308180, U.S. Pat. No. 6,413,539 and GB 998,794, which are hereby incorporated by reference.

The timing mechanism comprises the miniature test tube, the medium and the indicating substance. The timing mechanism lies dormant in the dry sampling tube. Following the addition of the liquid blood sample the indicating substance (lithium in the preferred embodiment) diffuses into the sample liquid medium at a steady state rate which will vary with and preferably be proportionate to the duration of time of exposure to the liquid. At the point of centrifugation the timing mechanism (being denser than blood) will be deposited at the bottom of the sampling tube with the sediment, and separated from the plasma and serum—effectively halting the diffusion of the indicating substance into the plasma or serum.

The concentration of indicating substance remaining in the serum or plasma will vary with and preferably be proportional to the duration of time that the timing mechanism was in contact with the liquid blood. The skilled person will appreciate that because a chemical rather than say electronic timing mechanism is employed it can be manufactured in bulk very cheaply, which is important if the mechanism is to achieve widespread use

FIG. 2 shows five example plots of lithium release from a cellulose fiber based timer device, showing lithium ion concentration against time. Readings were taken at 3, 6 and 48 hours. It can be seen that lithium ion concentration is approximately proportional to time up to 6 hours; the final data points show that a lithium concentration of approximately 1 mmol is reached after 48 hours, in this example.

The measuring mechanism will next be described. In a preferred embodiment, the concentration of lithium in the supernatant is proportional to the age of the sample, and in most analytical chemistry instruments, the concentration of Lithium can be directly measured. Depending on the data handling facilities of the analysers, the concentration may be converted into a reading of the age of the sample, and this result may be incorporated automatically into the laboratory report. This could indicate that the analysis was undertaken within an acceptable time and is therefore valid. Alternatively where it is shown that the analysis is delayed the assay results may be qualified or rejected. This facility is a significant advantage of embodiments of the invention as a laboratory may handle around 1000 samples each day making a manually read timer costly and often impractical.

About 5% of blood samples are “short”—that is only 1-2 mls instead of say 7 mls of blood is obtainable. There can be practical reasons for this, for example very difficult veins or a pediatric sample, and often the analysis can proceed anyway. However, if the sample is short (of low volume), then the accuracy of the timer can be affected. If the volume of the sample can be measured then one can, preferably automatically, correct for the inaccuracy. Thus in one enhancement of the basic system the timer device includes a known quantity of a second indicator substance/dye (or another metal ion) which is substantially fully released immediately on or soon after exposure to the liquid blood sample. The final concentration of the second indicator substance is inversely proportional to the volume of the sample. Thus by measuring the concentration of the second indicator the volume of the sample can be indirectly measured and, if necessary, a correction made to the sample age estimate,

To recap, broadly speaking we have described a method to improve the validity of results obtained from laboratory blood (and any other bodily fluid) testing by providing a means to ensue that the samples are not excessively aged prior to analysis. In many situations, the intended analyte is labile, and the duration and conditions of storage and transport of specimens prior to analysis can alter the result of the analysis. The present invention describes a timing device which is included in the sampling tube, and which is activated on the addition of the liquid sample. The timing device reaction is terminated on centrifugation of the sample or at the time of the analysis, whichever the sooner. The timing device generates a signal, which is proportionate to the age of the sample, which can be measured by the analysers.

Embodiments of the invention have been described with specific reference to measuring the age of blood samples but the skilled person will appreciate that similar techniques may be used to monitor the age of other bodily fluids, for example urine. Embodiments of the described methods and apparatus may be used with biological samples obtained from either the human or the animal body. It will further be appreciated that applications of the techniques are not restricted to biological samples for analysis and extend to other types of sample, for example blood for transfusion, depending on the indicating substance employed. (For blood for transfusion the age indicating substance should be safe for transfusion, for example a non-toxic dye).

Various alternatives to the above described techniques are possible. For example, a sample age monitor may comprise a two compartment container, where on exposure to liquid, a first compartment expands (for instance it could contain a hygroscopic substance such as cellulose), which compresses a second compartment, which contains the age indicator, thus expressing the age indicator by direct pressure.

Most blood sampling tubes are of the “Vacutainer” (trademark) variety nowadays. This means that they are precharged with a negative pressure (i.e. a partial vacuum) which is useful, because it ensures that the correct amount of blood is drawn into the sample chamber. A blood sample timer mechanism may thus lie dormant in the sampling tube and be activated by release of the vacuum. Thus a sample age monitor may comprise a two compartment timer container, where only, release of the vacuum, a first compartment shrinks to release or express tile contents of a second compartment. Another option for initiating the tinier mechanism, is to rely upon piercing of the blood sampling tube by insertion of the needle to disrupt a membrane in the timer.

No doubt many other effective alternatives will occur to the skilled person. It will be understood that the invention is not limited to the described embodiments and encompasses modifications apparent to those skilled in the art lying within the spirit and scope of the claims appended hereto.

1-28. (canceled)

29. A sample age monitor for monitoring the age of a biological sample, said sample age monitor comprising a release device to provide an age indicator into a medium substantially predictably such that an age estimate of said sample is determinable by measuring a level of said age indicator.

30. A sample age monitor as claimed in claim 29 wherein said medium comprises said sample, and wherein said age estimate of said sample is determinable by measuring a level of said age indicator in said sample.

31. A sample age monitor as claimed in claim 29 wherein said release device includes a carrier, storing a material which provides said age indicator, and wherein said release device comprises a container having an opening to provide said age indicator into said sample.

32. A sample age monitor as claimed in claim 29 wherein said age indicator comprises a metal ion.

33. A sample age monitor as claimed in claim 29 wherein said age indicator comprises a dye.

34. A sample age monitor as claimed in claim 29 wherein said carrier comprises one or more of cellulose fiber, a complex carbohydrate, a hydrocolloid, and a biodegradable polymer.

35. A sample age monitor as claimed in claim 29 wherein said release device is configured to release two substances at least one of which acts on the other to provide said age indicator.

36. A sample age monitor as claimed in claim 35 wherein said release device comprises one or more dissolving pills.

37. A sample age monitor as claimed in claim 29 wherein said release device comprises a volume change part configured to change volume on collection of said sample to provide said age indicator into said medium.

38. A sample age monitor as claimed in claim 29 wherein said release device comprises a membrane pierced on collection of said sample to provide said age indicator into said medium.

39. A sample age monitor as claimed in claim 29 wherein said release device is further configured to provide a predetermined quantity of a second indicator into said medium on collection of said sample.

40. A sample age monitor as claimed in claim 29 wherein said biological sample comprises blood.

41. A sample age monitor as claimed in claim 29 wherein said age estimate is accurate to better than 30 minutes over at least a six hour period.

42. A controlled release device for determining the age of a blood sample, the device being configured to release an age indicator into the blood sample on contact with the sample, and to substantially cease release of said age indicator into plasma of said blood sample after centrifugation of said sample.

43. A controlled release device as claimed in claim 42 wherein said device has an average density, after immersion in said blood sample, greater than that of cells of said sample.

44. A biological sample holder including the controlled release device of claim 42.

45. A biological sample holder including a timer mechanism to determine a duration of time between addition of a sample to said holder and centrifugation or laboratory analysis of the sample.

46. A biological sample holder as claimed in claim 45 wherein said timer mechanism is a chemical timer mechanism.

47. A method of determining an age for a biological sample, the method comprising:
containing the sample after collection together with an age indicator release device; and
determining a level of said age indicator to determine said age.

48. A method as claimed in claim 47 wherein said release device includes a carrier medium incorporating said age indicator or a precursor or generator of said age indicator.

49. A method as claimed in claim 48 wherein said release device comprises an enclosure having an indicator release portion for release of said age indicator or said age indicator precursor or generator into said sample.

50. A method as claimed in claim 47 wherein said biological sample comprises a blood sample.

51. A method as claimed in claim 47 wherein said age indicator comprises a metal ion.

52. A method as claimed in claim 47 wherein said age indicator comprises a dye.

53. A method as claimed in claim 47 wherein said release device has an average density, after immersion in said sample, greater than that of cells of said sample, and wherein said method further comprises centrifuging said sample prior to said age indicator level determining.

54. A method of assaying a biological sample including the method of claim 47 and wherein said age indicator level determining is performed in conjunction with said assaying.
